(19) United States
(12) Patent Application Publication
(10) Pub. No.: US 2008/0227191 A1
(43) Pub. Date: Sep. 18, 2008

(54) CELL CULTURE VESSEL AND CELL CULTURE APPARATUS

(75) Inventor: Mamoru Tsukada, Fujisawa-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/036,853

(22) Filed: Feb. 25, 2008

(30) Foreign Application Priority Data
Mar. 16, 2007 (JP) 2007-069054

(51) Int. Cl. C12M 1/00

(52) U.S. Cl. 435/308.1

(57) ABSTRACT

A vessel and an apparatus for culturing cells are provided in which a temperature difference is effected so that any position in the vessel can be brought into a temperature at which cells can be attached and a temperature at which cells cannot be attached, thereby freely selecting and recovering cells.

### BACKGROUND OF THE INVENTION

1. Field of the Invention

The present invention relates to a vessel and an apparatus used for culturing cells.

2. Related Background Art

A cell culture substrate using a temperature-responsive polymer has been conventionally known (see WO 93/03139). In addition, a method of removing cells from a separating material made of a temperature-responsive polymer without losing the functions of the cells has been known (see Japanese Patent No. 03,441,530). The method allows cells to attach to the polymer and causes a conformational change in the polymer to remove the cells therefrom. Further, WO 01/068799 discloses a method of co-culturing cells of different types by placing temperature-responsive polymers having different properties on different areas in predetermined patterns. The document indicates that the method makes it effective to use differences in properties of the different temperature-responsive polymers to selectively recover the cells of the same type.

Each of the above-mentioned conventional technologies has an excellent feature of causing less damage to cells as compared with the conventional cell-removal method using protease such as trypsin.

On the other hand, any conventional technology for freely selecting and detaching cells with the above temperature-responsive polymer in a more satisfactory manner has not been disclosed so far. For example, in the case of culturing certain stem cells whose differentiation fate is still undetermined, it has been desired to selectively remove or recover cells which can be identified by morphological characteristics or any marker from the above cells. Likewise, in the case of establishing transgenic cells, it has been desired to selectively recover cells whose transduction is completed. Further, just in the case of the passage of cells, the number of cells to be subjected to passage should be reduced for preventing the cells from reaching excessive confluence.

### SUMMARY OF THE INVENTION

The present invention is aimed at providing a vessel and an apparatus for culturing cells, in which a temperature difference is effected so that any position in the cell culture vessel can be brought into a temperature at which cells can be attached and a temperature at which cells cannot be attached, thereby freely selecting and recovering cells.

A cell culture vessel of the present invention is provided with a cell-attachment surface having a temperature-responsive polymer and includes a heating device for site-selectively heating the cell-attachment surface to perform a site-selective detachment of a cell from the cell-attachment surface.

A cell culture apparatus of the present invention includes a cell culture vessel provided with a cell-attachment surface having a temperature-responsive polymer, including a heating device for site-selectively heating the cell-attachment surface to perform site-selective detachment of a cell from the cell-attachment surface, and a control device for controlling site-selective heating of the cell-attachment surface by the heating device.

Further features of the present invention will become apparent from the following description of exemplary embodiments with reference to the attached drawings.

### BRIEF DESCRIPTION OF THE DRAWINGS

FIG. 1 is an explanatory diagram of a cell culture vessel of the present invention.

FIG. 2 illustrates an example in which a heating device of the cell culture vessel of the present invention is heating resistive elements.

FIG. 3A is a plan view of FIG. 2 and FIG. 3B is a cross-sectional view of FIG. 2.

FIG. 4 illustrates an example in which heating resistive elements are independently provided with TFTs in the cell culture vessel of the present invention.

FIG. 5A is a plan view of FIG. 4 and FIG. 5B is a cross-sectional view of FIG. 4.

FIG. 6 illustrates an example of a dielectric heating effect in the cell culture vessel of the present invention.

FIG. 7A is a plan view of FIG. 6 and FIG. 7B is a cross-sectional view of FIG. 6.

FIG. 8 illustrates an example of a cell culture apparatus of the present invention.

FIG. 9 illustrates an example of the cell culture apparatus of the present invention.

FIG. 10 illustrates the cell culture vessel of the present invention.

FIG. 11 illustrates the whole configuration of the cell culture apparatus of the present invention.

### DESCRIPTION OF THE EMBODIMENTS

A cell culture vessel according to the present invention includes a cell-attachment surface on which a culture solution, a washing fluid, etc. can be applied. The cell-attachment surface includes a temperature-responsive polymer, so the surface has a function of reversibly changing the adhesive property thereof to cells depending on temperature. Further, the cell culture vessel includes a heating device which can site-selectively heat the cell-attachment surface. Thus, the heating device is allowed to heat any site of the cell-attachment surface.

The cell culture apparatus of the present invention includes a cell culture vessel which is constructed as described above and a control device for instructing the heating device the cell culture vessel has to perform site-selective heating. The cell culture apparatus may further include a heating-site selection device for selecting the heating-site of the cell-attachment surface formed on the cell culture vessel. The control device instructs the heating device to heat the site selected by the heating-site selection device. The heating-site selection device may include a device for taking an image of the cell-attachment state on the cell-attachment surface.

Hereinafter, the present invention will be further described with reference to the accompanying drawings.

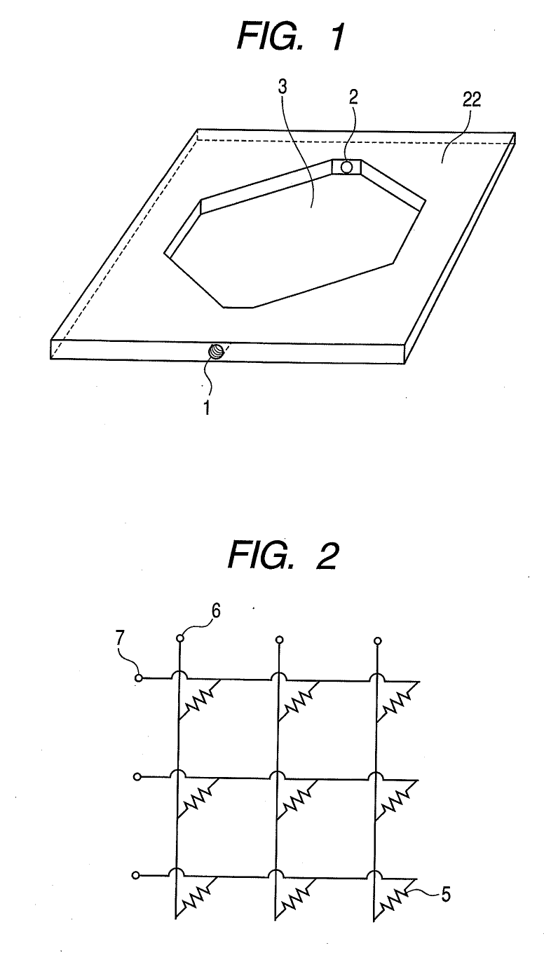

FIG. 1 is an explanatory diagram of a cell culture vessel of the present invention. The cell culture vessel has an inlet 1 and an outlet 2 for circulation of a culture solution or another buffer solution. The cell culture vessel has a bottom 3 as a cell-attachment surface provided with a temperature-responsive polymer. Further, a large number of heating devices 22, preferably transparent heating devices, are arranged in a lattice pattern over the cell-attachment surface to heat any position thereof. The top of the cell culture vessel may be opened or partially opened, or may be covered with a ceiling member to seal the inside of the vessel.

Any of the following may be used as the heating devices 22 which can site-selectively heat the desired position of the cell-attachment surface.

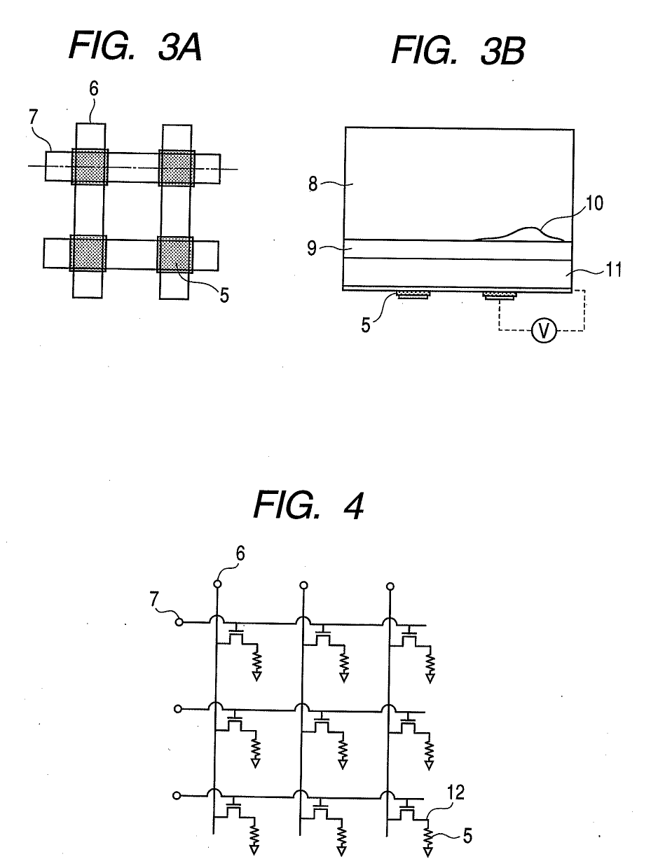

(1) FIG. 2 is an electric-wiring diagram illustrating an example of the heating device 22 in the case where heating resistive elements 5 in a 3×3 lattice pattern are two-dimensionally arranged at positions corresponding to the cell-attachment surface. Signal lines of a pair of row 7 and column 6 are selected to drive a heating resistive element 5 at the desired position, thereby allowing the position to be heated. The heating resistive elements 5 may be sequentially driven one by one. Alternatively, two or more heating resistive elements 5 may be simultaneously driven depending on circumstances. FIGS. 3A and 3B illustrate the actual configuration of the wiring. FIG. 3A shows that each heating resistive element 5 having high resistance is sandwiched between signal lines extending along row 7 and column 6. FIG. 3B illustrates a cross-sectional view of the cell culture vessel 11 taken along the broken line of FIG. 3A. In this figure, a cell 10 is cultured in a culture solution 8 on a temperature-responsive polymer 9.

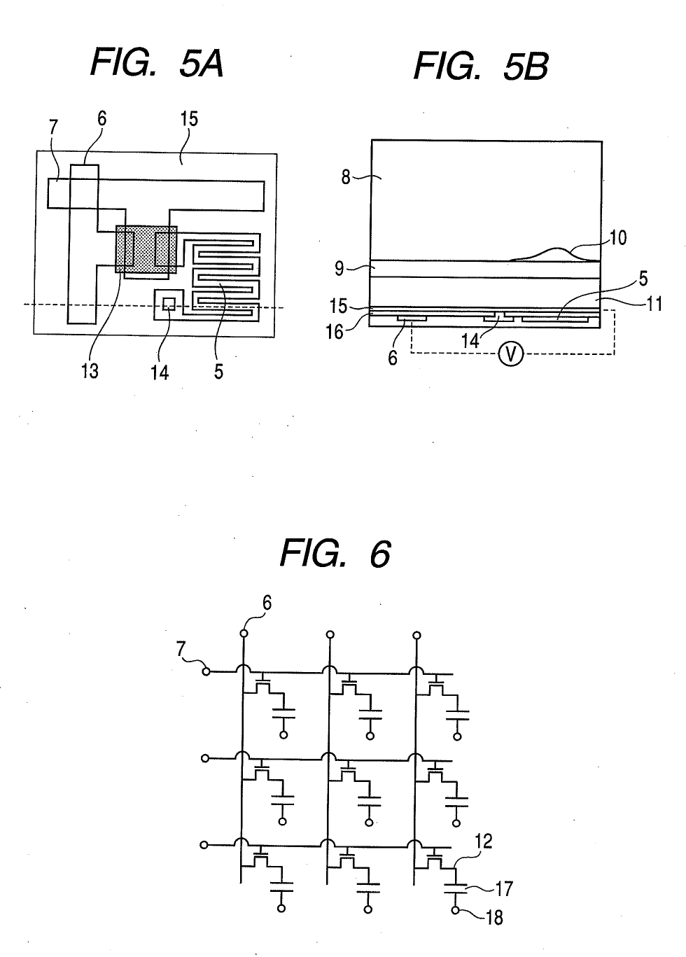

(2) FIG. 4 illustrates heating resistive elements 5 in a 3×3 lattice pattern and TFTs 12 provided thereto for independently driving the respective heating resistive elements 5. Thus, the heating resistive elements 5 can freely heat any position of the cell-attachment surface. FIG. 5A illustrates the actual configuration of the wiring. FIG. 5A shows that signal lines of row 7 and column 6 are connected to a gate electrode and a source electrode through a semiconductor thin film 13, respectively. The drain side is connected to the heating resistive element 5. The heating resistive element 5 can be heated when an electric current is allowed to flow in a ground layer 15 through a contact hole 14 formed on an insulating film 16. FIG. 5B illustrates a cross-sectional view of the cell culture vessel 11 taken along the broken line of FIG. 5A. In this figure, a cell 10 is cultured in a culture solution 8 on a temperature-responsive polymer 9.

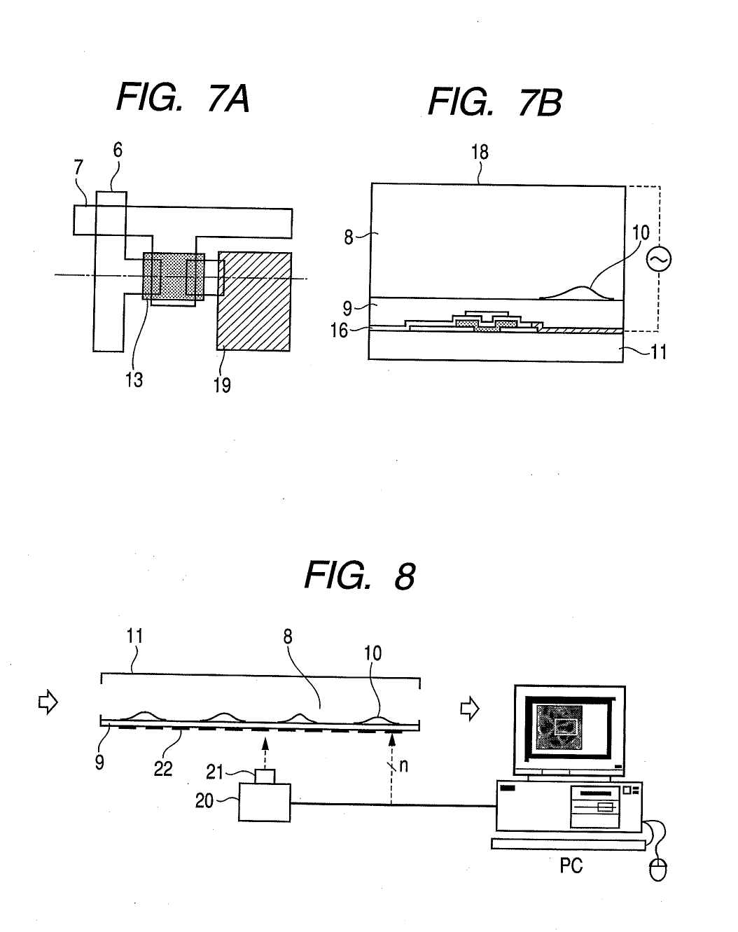

(3) FIG. 6 illustrates the heating devices 22 in a 3×3 lattice pattern having capacitors 17 and TFTs 12 for independently driving the respective capacitors 17. The principal configuration of the circuit is substantially the same as in a transparent liquid crystal display, and the capacitor 17 is connected to a counter electrode 18 of a liquid crystal. In this case, however, the content of the capacitor 17 is not a liquid crystal. FIG. 7B is a cross-sectional view taken along the broken line of FIG. 7A. As shown in FIG. 7B, a cell 10 is cultured in a culture solution 8 on a temperature-responsive polymer 9 placed between the counter electrode 18 and a pixel electrode 19. This position can be heated as a result of a dielectric heating effect when an alternating current is applied at the position. The voltage is approximately 10 volts or less and the frequency may be within the operating range of TFT.

Referring to FIG. 8, the vessel which can heat any position of the cell-attachment surface as described above is described in a case where a cell culture apparatus of the present invention is used.

A cell culture vessel 11 includes a layer formed of a temperature-responsive polymer 9 provided as a cell-attachment surface to culture cells. An observation image of cultured cells can be taken in a personal computer (PC) through a CCD camera 20 and a microscope unit 21. The microscope unit may be an inverted (optical/fluorescence) microscope unit. The user may select any position in the cell culture vessel by means of a mouse or a keyboard with reference to the obtained image. The selected cell position corresponds to the location of a cell to be detached (or left for subsequent passage culture). The temperature-responsive polymer causes phase transition from a liquid state to a gel state as the temperature is raised. The polymer in a gel state allows cells to be attached thereto, whereas the polymer in a liquid state allows the cells to be detached therefrom. Thus, the cells can be detached from the given position in the cell culture vessel by bringing about the temperature difference enough to effecting phase transition between the temperature of the selected position and the temperature of the nonselected position. In order to induce the temperature difference, the selected cell position is heated by the heating device 22 while circulating a culture solution, a physiological salt solution, or a phosphate buffer solution (PBS) at a temperature equal to or lower than the temperature at which the temperature-responsive polymer is liquidized. Cells remain attached at the position where the gel state is maintained. In contrast, cells can be detached at the position being cooled by the circulation of the culture solution. Thus, the cells are circulated along with the culture solution and are recovered in a given bottle or the like.

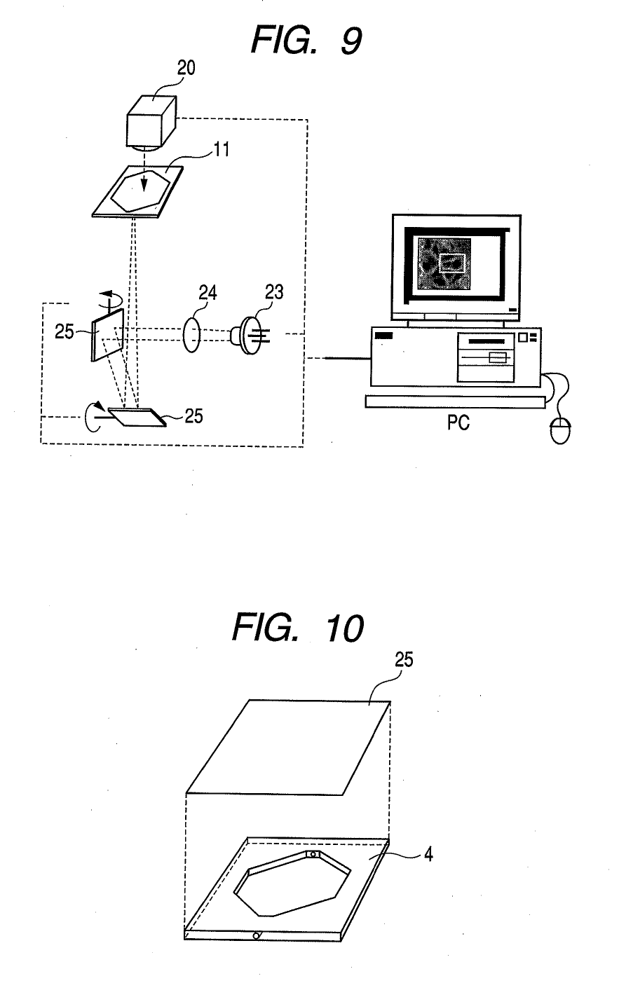

Further, for causing the above temperature difference, the heating device 22 may be installed in the outside of the cell culture vessel 11 instead of the inside thereof, whereby the cell culture apparatus of the present invention can be also configured. FIG. 9 illustrates the process of scanning a culture surface by condensing light rays from a near-infrared (or infrared) semiconductor laser 23 through a condensing lens 24. An area irradiated with the laser light is in a gel state, thereby retaining the ability to attach cells. A two-dimensional galvanometer mirror 25 may be used for scanning the culture surface. The ON/OFF control of the laser may be carried out in synchronization with the position of the culture surface. For example, the information about the selected position of the targeted cell is previously obtained using a mouse and the laser is then controlled on the basis of the positional information.

The temperature difference between the respective positions can be created by circulating the culture solution at a temperature equal to or lower than the temperature at which the temperature-responsive polymer is liquidized as described above.

It should be noted that infrared light may be generated by a combination of a tungsten lamp or a halogen lamp and a filter transparent to infrared light instead of using a laser. In addition, the cell culture vessel is configured so that the vessel can be irradiated with light when the heating is site-selectively carried out by light irradiation or the like. In addition, a portion made of a material to be heated by light irradiation is formed on the cell-attachment surface or the position corresponding thereto.

The procedure for generating a temperature difference can be described in the same manner using the following heat conduction equation even when the heating is performed by means of any of a heating resistive element, a dielectric heating effect and laser light irradiation.

Where “T” is a temperature, “t” is a time, “λ” is the thermal conductivity of a heat reservoir, “Cv” is the thermal capacity of the heat reservoir, and “Q” is a heat source, the heat conduction equation can be represented as follows:

$$\frac{\partial T}{\partial t} = \frac{λ}{Cv} {∇}^{2} T + Q \qquad [ Equation 1 ]$$

In a case where heat is not supplied from the heat source to the cell culture vessel, i.e., at the position of heating OFF, the equation is represented as follows:

$$Q = 0 , \\ thus \frac{\partial T}{\partial t} = \frac{λ}{Cv} {∇}^{2} T \qquad [ Equation 2 ]$$

When the culture solution is sufficiently circulated in the cell culture vessel and reaches a steady state, the equation is represented as follows:

$$\frac{\partial T}{\partial t} = 0 \qquad [ Equation 3 ]$$

Consequently, no temperature gradient occurs at the position of heating OFF, and hence, cells can be detached.

In contrast, when it is in a steady state at the position of heating ON, the equation is represented as follows:

$$- Q = \frac{λ}{Cv} {∇}^{2} T \qquad [ Equation 4 ]$$

Consequently, the amount of heat supplied is in an equilibrium state with a certain temperature gradient at the position of heating ON. Therefore, at the position of heating ON, the gel state can be retained by supplying heat.

Further, in the example of the cell culture apparatus illustrated in FIG. 8, a heating-site selection device includes a microscope unit 21, a CCD camera 20, and an image processing unit for displaying the image of the cell-attachment surface on a PC and specifying the heating position. In addition, the PC is provided with the function of controlling a device for instructing the heating device 22 to heat a given position on the cell-attachment surface of the cell culture vessel corresponding to the heating position specified on the screen.

### EXAMPLES

### Example 1

An example of a culture vessel of the present invention will be described below.

As illustrated in FIG. 10, an indium tin oxide (ITO) film with a thickness of about 300 nm is formed by sputtering on a predetermined area of the backside of a member 4 previously molded from an acrylic plate. A resist, AZ1500 (trade name, manufactured by AZ Electronic Materials Ltd.), is applied on the ITO film and then subjected to exposure and development. Subsequently, a signal line pattern of row 6 is formed on the resulting product by using an ITO etching solution (manufactured by Kanto Kagaku Co., Ltd.) and the resist is then removed from the film by washing with acetone. Further, another resist, ZPN1000 (trade name, manufactured by Tokyo Zairyo Co., Ltd.), is applied on the film, followed by exposure and development. A transparent conductive film coating composition in which oxide fine particles are dispersed, ECH-111 (trade name of Colcoat Co., Ltd.), is applied by spin-coating on the film and then irradiated with UV light to form a high-resistance film on the surface of the above film. The resist is removed from the film by a lift-off technology with acetone and ultrasonic cleaning, followed by forming a tile-like resistive element pattern on each lattice point (see FIG. 3A). Subsequently, a signal line pattern of column 7 is formed on the film. A resist, ZPN1000 (trade name, manufactured by Tokyo Zairyo Co., Ltd.), is applied on the film, followed by exposure and development. Subsequently, an ITO film with a thickness of about 300 nm is formed on the film by sputtering and then subjected to a lift-off technology with acetone and ultrasonic cleaning to remove the resist. Consequently, electrodes in a lattice pattern can be formed. In this case, a heating resistive element having high resistance with oxide fine particles dispersed therein is sandwiched between the electrodes.

Likewise, on an acrylic plate to be formed into a lid 25, an ITO film with a thickness of about 20 nm is formed by sputtering. The ITO film formed on the lid 25 can be used as a dew drop prevention heater when the vessel is not filled with a liquid. For connecting the lid 25 with the member 4 to allow electric current to flow through each of the signal lines of row 7 and column 6 arranged in a lattice pattern, an anisotropic conductive rubber with a pitch of 100 μm (manufactured by Fuji Polymer Industries, Co., Ltd.) is used. The lid 25, the member 4 and the anisotropic conductive rubber are superimposed and then bonded and fixed by a holder.

### Example 2

An example of a culture apparatus of the present invention will be described below.

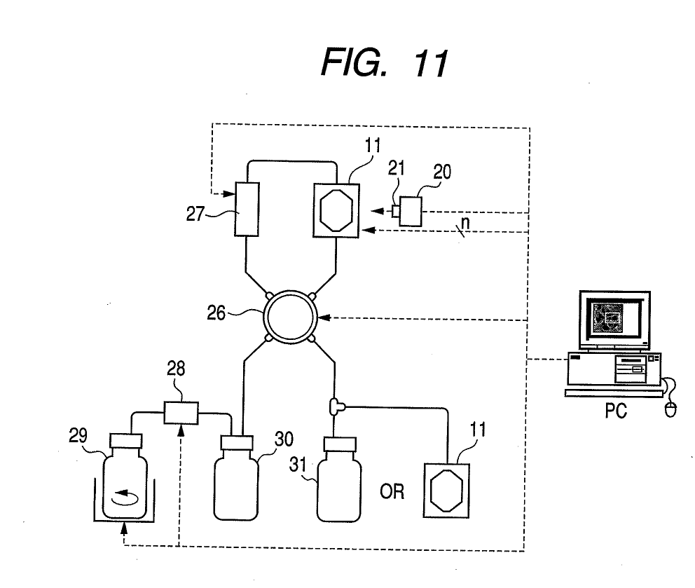

As illustrated in FIG. 11, a cell culture apparatus is provided with a PC including a control device and a heating-site selection device, and signals can be transmitted to all of the required devices through analog I/O and digital I/O built in the PC. On the other hand, the PC can take in a culture image in the cell culture vessel 11 from an inverted (optical/fluorescence) microscope unit 21 and a CCD camera 20. The area of the cell-attachment surface of the cell culture vessel 11 prepared in Example 1 is 400 mm² and 20 μl of a temperature-responsive gel, poly(N-isopropyl acrylamide) (Mn=20,000 to 25,000; manufactured by Aldrich Corp.), is then dispensed onto the bottom of the cell culture vessel 11. The gel is spread over the whole bottom surface and then left standing for about 30 minutes at room temperature. The excess liquid is removed by suction. Subsequently, 80 μl of a cell suspension (approximately 1×10⁵ cells/ml) is seeded in the gel. Then, the lid 25 is placed and fixed on the cell culture vessel 11. In a CO₂-generation bottle 29, 5% sodium bicarbonate solution is stirred with a stirrer, while the periphery of the CO₂-generation bottle 29 is heated at about 40° C. The generated CO₂ gas is fed into a culture-fluid supply bottle 30 by a pump 28 and the culture solution is then bubbled. In this case, the culture solution is previously placed in the culture-fluid supply bottle 30. The culture solution is absorbed by a direction-switching valve 26 having a pump, and the cell culture vessel 11 during culture can be constantly supplied with a fresh culture solution. As far as the culture solution does not deteriorate, the culture solution kept at 37° C. by a pump 27 provided with a Pertier controller can be constantly circulated through the direction-switching valve 26 provided with a pump and the cell culture vessel 11. During culture, the time-lapse shoot of the culture can be taken by the CCD camera 20. When the selective detachment of cells is requested, the culture solution being circulated and kept at 37° C. by the pump 27 provided with the Pertier controller is lowered to 20° C. Then, the cell-attached site desired to be retained in the attached state is selectively heated, while the cells detached by lowering the temperature of the culture solution are recovered in a waste fluid collecting bottle 31 or recovered in the cell culture vessel 11 to carry out additional culture of the cells.

The vessel and the apparatus of the present invention suitable for cell culture have the configurations for freely selecting and recovering cells by effecting a temperature difference on the temperature-responsive polymer so as to have a portion to be brought into a temperature at which cells can be attached and a portion to be brought into a temperature at which cells cannot be attached. Therefore, only the targeted cell or cell population subjected to differentiation induction or gene introduction can be selectively recovered and then subcultured. In addition, the cells can be automatically subjected to subculture without any manual operation if the subculture is random and non-selective passage culture.

Further, the cell culture vessel and cell culture apparatus of the present invention can be used in cell culture to satisfy the needs of cell isolation and extraction generated in relation to essential technological factors such as inheritable genetic modification or property modification of cells, differentiation, dedifferentiation and un-differentiation involved with stem cells, establishment of cells, establishment of cell lines, and immortalization of cells. In addition, the cell culture vessel can be used in continuous culture while being closed from the outside, making culture automation easy, and hence, can be applied in culture that requires a clean environment, such as in the production of medicines. Also, cells can be aligned as wanted when the cells are cultured using the temperature-responsive polymer in a liquid state and preventing the cells from being attached. Thus, the present invention may be available for the analysis of the interaction between cells of different types or the same type and a method of controlling cells.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2007-069054, filed Mar. 16, 2007, which is hereby incorporated by reference herein in its entirety.

What is claimed is:

1. A cell culture vessel provided with a cell-attachment surface having a temperature-responsive polymer, comprising a heating device for site-selectively heating the cell-attachment surface to perform site-selective detachment of a cell from the cell-attachment surface.

2. A cell culture apparatus comprising a cell culture vessel provided with a cell-attachment surface having a temperature-responsive polymer, which comprises:
a heating device for site-selectively heating the cell-attachment surface to perform site-selective detachment of a cell from the cell-attachment surface;
a cooling device for lowering temperature of a culture solution to temperature for allowing the cell to be detached from the cell-attachment surface; and
a control device for controlling site-selective heating of the cell-attachment surface by the heating device.

3. A cell culture apparatus according to claim 2, further comprising a heating-site selection device for selecting a heating site on the cell-attachment surface,
wherein the control device instructs the heating device to heat a site selected by the heating-site selection device.

4. A cell culture apparatus according to claim 3, wherein the heating-site selection device includes an image-taking device for taking an image of a cell attachment state on the cell-attachment surface.
